SEQUENCE LISTING (8 sequences)

SEQ ID NO: 1 - protein, 4 aa, Artificial
  Synthetic peptide with free cysteine for thiol linkage
YCYY

SEQ ID NO: 2 - protein, 4 aa, Artificial
  Synthetic peptide with free cysteine for thiol linkage
CYYY

SEQ ID NO: 3 - protein, 4 aa, Artificial
  Synthetic peptide with free cysteine for thiol linkage
YYYC

SEQ ID NO: 4 - protein, 7 aa, Artificial
  Synthetic SSTR binding domain
CFWKTCT

SEQ ID NO: 5 - protein, 11 aa, Artificial
  Synthetic SST analog
YCYYCFWKTCT

SEQ ID NO: 6 - protein, 11 aa, Artificial
  Synthetic SST analog
CYYYCFWKTCT

SEQ ID NO: 7 - protein, 11 aa, Artificial
  Synthetic SST analog
YYCYCFWKTCT

SEQ ID NO: 8 - protein, 14 aa, Homo sapiens
  DISULFID (3)..(14)
AGCKNFFWKTFTSC

(19) United States
(12) Patent Application Publication
(10) Pub. No.: US 2005/0118099 A1
(43) Pub. Date: Jun. 2, 2005

(54) Thiol-mediated drug attachment to targeting peptides

(76) Inventors: Gary R. Braslawsky, San Diego, CA (US); Paul Chinn, Carlsbad, CA (US)

Correspondence Address: THOMAS CAWLEY;Pillsbury Winthrop, LLP, 1600 Tysons Blvd., McLean, VA, 22102, US

(21) Appl. No.: 10/796,158

(22) Filed: Mar. 10, 2004

Related U.S. Application Data
(60) Provisional application No. 60/452,928, filed on Mar. 10, 2003.

(51) Int. Cl. A61K 51/00; A61K 49/00

(52) U.S. Cl. 424/1.49; 424/9.34; 530/311

(57) ABSTRACT

Compositions and methods for thiol-specific attachment of therapeutic and diagnostic agents to somatostatin and other targeting peptides.

### PRIORITY INFORMATION

Priority is claimed to U.S. Provisional Patent Application No. 60/452,928, filed Mar. 10, 2003, which is incorporated herein in its entirety.

### FIELD OF THE INVENTION

The present invention generally relates to methods for site-specific attachment of drugs to peptides, and compositions produced by such methods. More specifically, the present invention relates to thiol-mediated drug attachment to somatostatin peptides, the resultant drug/peptide complexes, and uses thereof.

Table of Abbreviations

| | |
|---|---|
| AE | Auristatin E |
| AEB | Auristatin E derivative |
| AEBL | maleimido derivative of AEB |
| AR42J | SSTR-positive rat pancreatic carcinoma cells |
| COS-7 | SSTR-negative monkey kidney cells |
| CP1 | somatostatin analog |
| DMF | dimethyl formamide |
| DTPA | diethylenetriaminepentaacetic acid |
| FKMMAE | Auristatin E derivative |
| HPLC | high performance liquid chromatography |
| IC₅₀ | inhibitory concentration 50% |
| IMR-32 | SSTR-positive human neuroblastoma cells |
| LS174T | SSTR-negative human colon carcinoma cells |
| MEM-MX-DTPA | maleimido derivative of MX-DTPA |
| MTD | maximal tolerable dose |
| MTT | 3-(4,5-dimethylthiazol-2-yl)-2,5-diphenyl |
| | tetrazolium bromide |
| MX-DTPA | DTPA derivative |
| SCN | thiocyanate |
| SST | somatostatin |
| SSTR | somatostatin receptor |

### DESCRIPTION OF RELATED ART

Tumor-specific binding agents can be used for tumor diagnosis and tumor-specific drug delivery. Existing tumor-specific binding agents include regulatory peptides, which bind to high affinity receptors that are overexpressed in many tumors. These peptides are particularly useful for in vivo targeting of therapeutics and/or diagnostic agents because they are small diffusible molecules that bind to surface-expressed receptors. The high-affinity receptors are also present in other tissues, however, rapid cycling of the receptors in tumor cells offers the potential for differential peptide uptake when compared to normal tissues. As one example, high-affinity somatostatin (SST) binding sites are abundantly expressed in most endocrine tumors, and radiolabeled SST analogs have been successfully used for diagnosis and therapy of such tumors. See e.g., Weckbecker et al. (1993) Pharmacol Ther 60:245-64; Srkalovic et al. (1990) J Clin Endocrinol Metab 70:661-9; Buscail et al. (1995) Proc Natl Acad Sci USA 92:1580-4; Reubi et al. (1995) J Clin Endocrinol Metab 80:2806-14; Reubi et al. (1996) Metabolism 45:39-41; Buscail et al. (1994) Proc Natl Acad Sci USA 91:2315-9; Patel (1997) J Endocrinol Invest 20:348-67; Patel et al. (1995) Life Sci 57:1249-65; Bruns et al. (1994) Ann N Y Acad Sci 733:138-46; Reisine & Bell (1995) Endocr Rev 16:427-42; Krenning et al. (1993) Eur J Nucl Med 20:716-31; Plonowski et al. (2002) Int J Oncol 20:397-402; Szepeshazi et al. (2001) Clin Cancer Res 7:2854-61; Kiaris et al. (2001) Eur J Cancer 37:620-8; Plonowski et al. (2000) Cancer Res 60:2996-3001; Kahan et al. (1999) Int J Cancer 82:592-8; Plonowski et al. (1999) Cancer Res 59:1947-53.

Despite these advances, the use of peptide analogs in diagnosis and therapy is limited by the relatively short half-life of these analogs in vivo. See e.g., Decristoforo & Mather (1999) Nucl Med Biol 26:389-96. For example, conjugation of somatostatin analogs via the terminal amino group using phenylisothiocyanate moieties results in Edman degradation of the conjugate and loss of the chelating moiety (e.g., for radioisotopes) or the attached drug.

Thus, there exists a long-felt need in the art for targeting peptides and peptide analogs that have improved stability following conjugation. To meet this need, the present invention provides methods and compositions for thiol-specific attachment to targeting peptides, including somatostatin analog peptides, having stability suitable for in vitro and in vivo uses.

### SUMMARY OF THE INVENTION

The present invention provides peptide analogs for thiol-specific drug attachment, and methods for using the same. Modification of existing peptide ligands so as to include sequences for thiol-specific drug attachment, as disclosed herein, enables preparation of peptides using phenylisothionate chemistries to attach drugs, chelators, or isotopes, which peptide conjugates have improved in vitro and in vivo stability. This method is generally applicable and useful for all peptides where modification of the carboxyl end of the peptide results in reduced binding to the target.

A representative peptide analog is a somatostatin analog of the formula (A-B), wherein A is cysteine, or a peptide chain comprising one or more cysteine residues and is suitable for conjugation to a drug (e.g., a radioisotope) or chelator via a thiol linkage to the one or more cysteine residues; and B is a naturally occurring or synthetic somatostatin peptide that specifically binds to a somatostatin receptor. Representative somatostatin analogs of the formula (A-B) are set forth as SEQ ID NOs: 5-7.

With reference to a peptide analog of the formula (A-B), as described herein, the A peptide includes at least one cysteine, which mediates thiol-specific drug attachment. Thus, in alternate embodiments of the invention, the A peptide includes one cysteine or multiple cysteines. If A includes a terminal cysteine, the terminal cysteine is N-blocked and an SCN reagent is used. Representative A peptides are set forth as SEQ ID NOs:1-3.

Where a chelator is used, the chelator mediates binding of a drug (e.g., a radioisotope) to the somatostatin analog at the one or more cysteine residues. Thus, thiol-specific drug attachment to a peptide analog can be direct or indirect, i.e. via a chelator. The present invention employs a chelator, which is a maleimido derivative of DTPA (MEM-MX-DTPA), useful in preparing the peptide analogs of the invention.

The peptide analogs of the present invention are suitable for thiol-specific attachment via a free cysteine. The thiol linkage can be a stable linkage, for example a thioether linkage. Alternatively, as desired for a particular application, the thiol linkage can be labile or hydrolyzable, such as a disulfide bond, an acid-labile linkage (e.g., a hydrazone bond), or an enzyme-labile linkage.

With reference to a somatostatin analog of the formula (A-B), the B peptide is any somatostatin peptide, i.e., any peptide that specifically binds to a somatostatin receptor, such as a human somatostatin receptor (SSTR). The somatostatin peptide mediates binding of the analog to SSTR-expressing cells. A representative somatostatin peptide is set forth as SEQ ID NO:4.

To increase the avidity of a peptide analog binding to its cognate receptor, the present invention further provides compositions comprising a matrix to which a plurality of peptide analogs of the invention are bound. Representative matrices include but are not limited to those matrices made of polyethylene glycol, polydextrans, cyclodextrins, polylysines, and the like. Where the peptide analogs are bound via a thiol linkage to a drug or chelator, the drug or chelator is also bound to the matrix. Alternatively, drugs and peptide analogs can each be attached directly to the matrix.

The peptide analogs of the invention are suitable for conjugation with any drug, including a therapeutic agents and diagnostic agents, which is capable of forming a thiol linkage. Representative therapeutic agents include radioisotopes, cytotoxins (e.g., a tubulin inhibitor), therapeutic genes, immunostimulatory agents, anti-angiogenic agents, and chemotherapeutic agents. Representative diagnostic agents include detectable labels, particularly those that are detectable in vivo, for example by using magnetic resonance imaging, scintigraphy, ultrasound, or fluorescence.

In a representative embodiment of the invention, a peptide analog is bound to a radioisotope. For therapeutic applications, useful radioisotopes include α-emitters, β-emitters (e.g., ⁹⁰yttrium), and auger electrons. For diagnostic applications, useful radioisotopes include positron emitters and y-emitters (e.g., ¹¹¹indium or ¹³¹iodine). Chelators such as maleimido derivatives of DTPA or a DTPA analog can mediate attachment of radioisotopes to targeting peptides of the invention.

The present invention further provides methods for using the peptide analogs as targeting peptides in a subject, including mammalian and human subjects. Thus, a peptide analog of the invention can bind to a cognate receptor in vivo. For example, a somatostatin analog of the invention specifically binds to one or more somatostatin receptors in vivo. This binding is the basis of diagnostic and therapeutic methods in mammals, including humans.

Thus, also provided are methods for detecting SSTR-positive cells in vivo via administration of a peptide analog of the invention. In a representative embodiment of the invention, the method comprises: (a) administering to the subject a composition comprising a somatostatin analog of the formula (A-B), wherein A is cysteine, or a peptide chain comprising one or more cysteine residues, wherein A is bound to the one or more cysteines via a thiol linkage, and wherein B is a somtaostatin peptide; and (b) detecting the detectable label, whereby SSTR-positive cells are detected.

Also provided are methods for the treatment of SSTR-associated diseases and disorders. In a representative embodiment of the invention, the method comprises administering to a subject in need of such treatment a composition comprising a somatostatin analog of the formula (A-B), wherein A is cysteine, or a peptide chain comprising one or more cysteine residues, wherein a therapeutic agent is bound to A via thiol linkage to the one or more cysteine residues, and wherein B is a somatostatin peptide, whereby an SSTR-associated disease or disorder is treated.

### BRIEF DESCRIPTION OF THE DRAWINGS

FIG. 1 is a line graph depicting competitive binding of Indium-111-octreotide to IMR-32 membranes in the presence of unlabeled octreotide (◯), CP1 (□), or CP1-AEBL (⋄). Competitive binding is indicated as the percent binding relative to a control level of binding (competitor not present). CP1 and CP1-AEBL inhibit Indium-111-octreotide to a similar extent as unlabeled octreotide (octreotide IC₅₀˜3 nM, CP1 IC₅₀˜2 nM, and CP1-AEBL IC₅₀˜2 nM).

FIGS. 2A-2B are line graphs depicting in vitro cytotoxicity induced by AEB (◯) and CP1-AEBL (□). In SSTR-positive IMR-32 cells, the CP1-AEBL conjugate was 100-fold less potent than the free drug, AEB (FIG. 2A). In SSTR-negative COS-7 cells, negligible cytotoxicity was observed in the presence of the CP1-AEBL conjugate (FIG. 2B). AEB induced showed a similar background level of cytotoxicity in both IMR-32 cells and COS-7 cells.

FIGS. 3A-3B are line graphs depicting tumor growth inhibition in an IMR-32 mouse xenograft model following administration of AE (◯), 1×CP1-FKMMAE (□), or 3×CP1-FKMMAE (⋄). The control sample depicts uninhibited tumor growth (X). Arrows indicate the times of administration, as described in Example 5. FIG. 3A shows a reduction in mean tumor volume, which was greatest in response to 3×CP1-FKMMAE. FIG. 3B shows that mean mouse weight slightly increased during the course of the study and was substantially similar among all treatment groups.

FIG. 4 is a line graph depicting growth hormone levels in an IMR-32 mouse xenograft model following administration of AE (◯), 1×CP1-FKMMAE (□), or 3×CP1-FKMMAE (⋄). The control sample depicts growth hormone levels in the absence of treatment (X). Arrows indicate the times of administration, as described in Example 5. Serum growth hormone levels were determined by ELISA to assess potential toxicity to the pituitary gland. The relative stability of growth hormone levels during the course of the study indicated the specificity of the anti-tumor response shown in FIG. 3A.

### DETAILED DESCRIPTION OF THE INVENTION

I. Definitions

While the following terms are believed to be well understood by one of ordinary skill in the art, the following definitions are set forth to facilitate explanation of the invention.

The term “somatostatin peptide” refers to a peptide that specifically binds to a somatostatin receptor (SSTR), such as a somatostatin receptor expressed on a cell. Native somatostatin is a peptide having an amino acid sequence set forth as SEQ ID NO:8. Thus, the term “somatostatin peptide” includes the full-length sequence of SEQ ID NO:8, as well as fragments thereof that specifically bind to a somatostatin receptor.

The term “somatostatin peptide” also encompasses cyclic and linear peptide analogs. Many such peptide analogs have been described in the art and can be used in accordance with the present invention, for example in U.S. Pat. Nos. 6,465,613; 6,001,801; 5,770,687; 5,750,499; 5,708,135; 5,633,263; 5,620,675; 5,597,894; 5,716,596; 5,633,263; 5,411,943; 5,073,541; 4,904,642; 4,871,717; 4,853,371; 4,485,101; each of which is hereby incorporated by reference. A representative somatostatin peptide is set forth as SEQ ID NO:4.

The term “somatostatin receptor,” which is abbreviated herein as SSTR, refers to a mammalian somatostatin receptor, such as a human somatostatin receptor. SSTRs are known in the art, and can be readily synthesized, recombinantly expressed, and/or detected using conventional techniques in the art. The term “SSTR” encompasses SSTR subtypes, i.e. SSTR1, SSTR2, SSTR3, SSTR4, and SSTR5, which are structurally related integral membrane glycoproteins having similar binding properties.

The term “binding” refers to an affinity between two molecules, for example, a peptide ligand and a receptor. As used herein, “binding” means a preferential binding of one molecule for another in a mixture of molecules. The binding of a ligand to a receptor can be considered specific if the binding affinity is about 1×10⁴ M⁻¹ to about 1×10⁶ M⁻¹ or greater.

The phrase “specifically (or selectively) binds”, as used herein to describe the binding capacity of a peptide, refers to a binding reaction which is determinative of the presence of the protein in a heterogeneous population of proteins and other biological materials. The phrase “specifically binds” also refers to selectively targeting, as described herein below.

The term “somatostatin-associated,” as used herein to describe a disease or disorder treatable by the disclosed peptide analogs, refers to a condition characterized by abnormal SSTR expression and/or function. Abnormal SSTR expression refers to somatostatin receptor expression on the surface of a specific normal cell type, which expression is at a level significantly greater than a surface expression level normally associated with that specific normal cell type. For example, tumors characterized as neuroblastomas aberrantly express somatostatin receptors in that the cells of a neuroblastoma have a higher level of somatostatin receptor surface expression than the nerve tissue from which the neuroblastoma was derived. Abnormal SSTR function refers to conditions of abnormally elevated or abnormally suppressed signaling via SSTR. Such conditions are characterized, for example, by abnormal production of a somatostatin regulatable factor(s), which production is significantly greater than production of that same factor in the absence of the condition. Acromegaly, which is associated with over production of the somatostatin-regulatable factor, growth hormone and insulin-like growth factor-1, is an example of such a condition.

The term “drug” as used herein refers to any substance having biological or detectable activity. Thus, the term “drug” includes a pharmaceutical agent, a diagnostic agent, or a combination thereof. The term “drug” also includes any substance that is desirably delivered to cells expressing a receptor to which a peptide analog of the invention specifically binds (e.g., SSTR⁺ cells).

The term “about”, as used herein when referring to a measurable value such as an amount, a binding affinity, etc., is meant to encompass variations of ±20% or ±10%, or ±5%, or ±1%, or ±0.1% from the specified value, as such variations are appropriate to perform the disclosed methods.

The terms “a,” “an,” and “the” are used in accordance with convention in the art to refer to one or more.

II. Peptide Analogs

The peptide analogs of the invention are designed so as to provide site-specific drug attachment to the peptide via a thiol linkage. In general, a site for drug attachment to the peptide is selected as a site removed from residues involved in ligand binding, for example, residues involved in binding to a target molecule in vivo.

In one embodiment of the invention, thiol-mediated drug attachment is effected at an interior peptide site. The term “interior” as used herein to describe a site for thiol-mediated attachment, refers to a non-terminal site, i.e. a site other than at the carboxyl or amino terminus of the molecule. An interior thiol typically comprises a thiol functional group on a non-terminal amino acid of a peptide chain. An interior thiol functional group can also comprise a thiol group of a terminal cysteine, wherein the terminal amino or carboxyl group is blocked from derivatization.

The disclosed analogs show improved stability as required for in vitro and in vivo applications. In particular, existing somatostatin analogs, which employ drug attachment at either the carboxyl or amino terminus of the analog using phenylisothiocyanate chemistries, have limited applicability because they are susceptible to Edmann degradation. The peptide analog design disclosed herein is also advantageous in that it preserves a “free” or unmodified amino terminus, which can be used for attachment of additional drugs and/or labels.

Peptide analogs of the invention are of the formula (A-B), wherein A is cysteine, or a peptide chain comprising one or more cysteine residues and is suitable for conjugation to a drug or chelator via a thiol linkage to the one or more cysteine residues; and B is a targeting peptide. The term “targeting peptide” is used herein to generally describe low molecular weight peptides that specifically bind to cognate receptors.

The disclosed methods are particularly relevant to conjugation of drugs/chelators to other low molecular weight peptides that show high affinity binding, for example vasointestinal peptide (VIP), bombesin, pituitary adenylate cyclase activating polypeptide (PACAP), Substance P, enkephalins, neurokinins, and derivatives and receptor binding fragments thereof. These peptides, and their binding to cognate receptors, are well characterized. Thus, following a review of the disclosure herein, one skilled in the art could readily prepare peptide analogs having interior sites for thiol-mediated attachment of drugs/chelators.

Representative analogs of the invention are described in the Examples. Example 1 describes a somatostatin analog bound to a model organic drug (Auristatin E) and to a radioisotope (Indium-111). These analogs represent exemplary embodiments of the present invention, and the novel compositions disclosed herein are not intended to be limited to these particular embodiments.

II.A. General Considerations

A binding peptide or peptide analog of the present invention can be subject to various changes, substitutions, insertions, and deletions where such changes provide for certain advantages in its use. Thus, the term “peptide” encompasses any of a variety of forms of peptide derivatives, that include amides, conjugates with proteins, cyclized peptides, polymerized peptides, conservatively substituted variants, analogs, fragments, peptoids, chemically modified peptides, and peptide mimetics.

Peptides of the invention can comprise naturally occurring amino acids, synthetic amino acids, genetically encoded amino acids, non-genetically encoded amino acids, and combinations thereof. Peptides can include both L-form and D-form amino acids.

Representative non-genetically encoded amino acids include but are not limited to 2-aminoadipic acid; 3-aminoadipic acid; P-aminopropionic acid; 2-aminobutyric acid; 4-aminobutyric acid (piperidinic acid); 6-aminocaproic acid; 2-aminoheptanoic acid; 2-aminoisobutyric acid; 3-aminoisobutyric acid; 2-aminopimelic acid; 2,4-diaminobutyric acid; desmosine; 2,2′-diaminopimelic acid; 2,3-diaminopropionic acid; N-ethylglycine; N-ethylasparagine; hydroxylysine; allo-hydroxylysine; 3-hydroxyproline; 4-hydroxyproline; isodesmosine; allo-isoleucine; N-methylglycine (sarcosine); N-methylisoleucine; N-methylvaline; norvaline; norleucine; and ornithine.

Representative derivatized amino acids include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups can be derivatized to form salts, methyl and ethyl esters or other types of esters or hydrazides. Free hydroxyl groups can be derivatized to form O-acyl or O-alkyl derivatives. The imidazole nitrogen of histidine can be derivatized to form N-imbenzylhistidine.

The term “conservatively substituted variant” refers to a peptide, e.g., a somatostatin peptide or somatostatin peptide analog set forth as SEQ ID NO:4-7, comprising an amino acid in which one or more residues have been conservatively substituted with a functionally similar residue and which displays the targeting activity as described herein. The phrase “conservatively substituted variant” also includes peptides wherein a residue is replaced with a chemically derivatized residue.

Examples of conservative substitutions include the substitution of one non-polar (hydrophobic) residue such as isoleucine, valine, leucine or methionine for another; the substitution of one polar (hydrophilic) residue for another such as between arginine and lysine, between glutamine and asparagine, between glycine and serine; the substitution of one basic residue such as lysine, arginine or histidine for another; or the substitution of one acidic residue, such as aspartic acid or glutamic acid for another.

Peptides of the present invention also include peptides comprising one or more additions and/or deletions or residues relative to the sequence of a peptide whose sequence is disclosed herein, so long as the requisite targeting activity and/or thiol-specific drug attachment sites of the peptide are maintained. The term “fragment” refers to a peptide comprising an amino acid residue sequence shorter than that of a peptide disclosed herein.

Additional residues can also be added at either terminus of a peptide for the purpose of providing a “linker” by which the peptides of the present invention can be conveniently affixed to a label or solid matrix, or carrier. Amino acid residue linkers are usually at least one residue and can be 40 or more residues, more often 1 to 10 residues, but do alone not constitute peptide analogs having receptor binding activity. Typical amino acid residues used for linking are tyrosine, cysteine, lysine, glutamic and aspartic acid, or the like. In addition, a peptide can be modified by terminal-NH₂ acylation (e.g., acetylation, or thioglycolic acid amidation) or by terminal-carboxylamidation (e.g., with ammonia, methylamine, and the like terminal modifications), or cyclized. Terminal modifications are useful, as is well known, to reduce susceptibility by proteinase digestion, and therefore serve to prolong half life of the peptides in solutions, particularly biological fluids where proteases can be present.

The term “peptoid” as used herein refers to a peptide wherein one or more of the peptide bonds are replaced by pseudopeptide bonds including but not limited to a carba bond (CH₂—CH₂), a depsi bond (CO—O), a hydroxyethylene bond (CHOH—CH₂), a ketomethylene bond (CO—CH₂), a methylene-ocy bond (CH₂—O), a reduced bond (CH₂—NH), a thiomethylene bond (CH₂—S), a thiopeptide bond (CS—NH), and an N-modified bond (—NRCO—). See e.g., Corringer et al. (1993) J Med Chem 36:166-72, Garbay-Jaureguiberry et al. (1992) Int J Pept Protein Res 39:523-7, Tung et al. (1992) Pept Res 5:115-8, Urge et al. (1992) Carbohydr Res 235:83-93, and Pavone et al. (1993) Int J Pept Protein Res 41:15-20.

The term “peptide mimetic” as used herein refers to a ligand that mimics the biological activity of a reference peptide, by substantially duplicating the targeting activity of the reference peptide, but it is not a peptide or peptoid. A peptide mimetic typically has a molecular weight of less than about 700 daltons.

II.B. Somatostatin Analogs

Somatostatin analogs are described as representative peptide analogs of the invention. A somatostatin analog is described as having the formula (A-B), wherein A is cysteine, or a peptide chain comprising one or more cysteine residues and is suitable for conjugation to a drug or chelator via a thiol linkage to the one or more cysteine residues; and B is a somtaostatin peptide. Representative somatostatin analogs of the formula (A-B) are set forth as SEQ ID NOs: 5-7.

The A peptide includes at least one cysteine, which mediates thiol-specific drug attachment. Thus, in alternate embodiments of the invention, the A peptide includes one cysteine or multiple cysteines. Representative A peptides are set forth as SEQ ID NOs: 1-3.

In a somatostatin analog of the formula (A-B), the B peptide is any somatostatin peptide, i.e., any peptide that specifically binds to a somatostatin receptor, such as to a human somatostatin receptor. A somatostatin analog of the invention can include a somatostatin peptide, wherein in the carboxyl terminus has been modified to an alcohol or amide to improve in vivo stability. Alternatively, a somatostatin analog can include a somatostatin peptide with an unmodified carboxyl terminus (i.e., in its carboxylic acid form), for example, where such structure improves tumor uptake and hastens blood clearance. See e.g., U.S. Pat. No. 5,830,431. A representative somatostatin peptide is set forth as SEQ ID NO:4.

II.C. Thiol Linkages

The peptide analogs of the present invention are suitable for thiol-specific attachment via a free cysteine. Thiol-specific drug attachment to a peptide analog can be direct or indirect, i.e. via a chelator. The present invention employs a chelator, MX-DTPA, useful in preparing the peptide analogs of the invention. The maleimido derivatives of MX-DTPA chelator is reactive with thiol groups of the peptide analog (i.e., SH groups of one or more free cysteines) to form a thioether linkage. When using MEM-MX-DTPA, the reaction conditions should have a pH of less than about 7.5 to preclude reactivity with amino (—NH₂) groups.

The thiol attachment methods of the present invention are generally applicable to the attachment of drugs/chelators to regulatory and targeting peptides, and are not intended to be limited to somatostatin receptors. MEM-MX-DTPA is suitable for attachment to a free thiol of any regulatory or targeting peptide.

The thiol linkage can be a stable linkage, for example as a thioether linkage. Thus, in one embodiment of the invention, a drug or chelator is functionalized with a thiol reactive group (e.g., a maleimido group) that provides a stable thioether linkage. Optionally, a drug can comprise a cleavable site, such that a portion of the drug can be released from the peptide. Representative cleavable sites include acid-labile and enzyme-labile sites.

In another embodiment of the invention, as desired for a particular application, the thiol linkage can be labile. For example, the drug or chelator is functionalized with a thiol group enabling formation of a disulfide bond with the peptide. A conjugate so prepared is redox active, such that it is stable in the serum and is released upon entry into the reducing environment of the cell cytosol.

II.D. Drugs

The peptide analogs of the invention are suitable for conjugation with any drug, capable of forming a thiol linkage. Representative therapeutic drugs include radioisotopes, cytotoxins (e.g., a tubulin inhibitor), therapeutic genes, immunostimulatory agents, anti-angiogenic agents, and chemotherapeutic agents. Representative diagnostic drugs include detectable labels that can be detected in vivo, for example by using magnetic resonance imaging, scintigraphic imaging, ultrasound, or fluorescence.

In a representative embodiment of the invention, a peptide analog is bound to a radioisotope, which is useful for therapeutic and/or diagnostic applications depending on the selection of the radioisotope. Radioisotopes useful for radiotherapy include but are not limited to high energy radioisotopes, such as α-emitters, β-emitters, and auger electrons. Radioisotopes useful for diagnostic applications include but are not limited to positron emitters and γ-emitters.

A somatostatin analog, which includes a drug bound via a thiol-specific linkage, can further be iodinated, for example on a tyrosine residue of the analog, to facilitate detection or therapeutic effect of the analog. Iodination methods are known in the art, and representative protocols can be found, for example, in Krenning et al. (1989) Lancet 1:242-4 and in Bakker et al. (1990) J Nucl Med 31:1501-9.

II.E. Binding Properties of Peptide Analogs

With reference to a somatostatin analog of the formula (A-B), the B peptide is any somatostatin peptide, i.e., any peptide that specifically binds to a somatostatin receptor, such as to a human somatostatin receptor (SSTR). Representative somatostatin peptides are set forth as SEQ ID NOs: 4 and 8. The somatostatin peptide mediates binding of the analog to SSTR-expressing cells. Representative methods for determining binding of a somatostatin analog to SSTR and to SSTR-expressing cells are described in Examples 2-3.

An SSTR-positive cell can comprise a cell expressing a somatostatin receptor of any subtype. In one embodiment of the invention, a somatostatin analog can specifically bind to one type of a somatostatin receptor (e.g., somatostatin receptor type 2) but does not substantially bind to a second type of somatostatin receptor (e.g., somatostatin receptor type 5). In another embodiment of the invention, a somatostatin analog can specifically bind multiple somatostatin receptor types (e.g., somatostatin receptor type 2 and type 4).

To increase binding avidity, the present invention further provides compositions comprising a carrier, which encapsulate or bind to a plurality of peptide analogs. Where drugs are bound to the peptide analogs via a thiol-specific linkage, the drugs are thereby also associated with the carrier. Alternatively, drugs and peptide analogs can each be attached directly to the matrix. The peptide analogs used to prepare a carrier/peptide analog composition can be identical or non-identical, i.e. wherein the peptide analogs include different drugs/chelators. Different peptide analogs can also comprise different peptides that bind to the same receptor.

Representative carriers include a microcapsule, for example a polymeric micelle or conjugate (Goldman et al. (1997) Cancer Res 57: 1447-51; U.S. Pat. Nos. 4,551,482, 5,714,166, 5,510,103, 5,490,840, and 5,855,900), a microsphere or a nanosphere (Manome et al. (1994) Cancer Res 54: 5408-13; Saltzman et al. (1997) Adv Drug Deliv Rev 26: 209-230), a glycosaminoglycan (U.S. Pat. No. 6,106,866), a fatty acid (U.S. Pat. No. 5,994,392), a fatty emulsion (U.S. Pat. No. 5,651,991), a lipid or lipid derivative (U.S. Pat. No. 5,786,387), collagen (U.S. Pat. No. 5,922,356), a polysaccharide or derivative thereof (U.S. Pat. No. 5,688,931), a nanosuspension (U.S. Pat. No. 5,858,410), and a polysome (U.S. Pat. No. 5,922,545).

For preparation of compositions with increased avidity of peptide analog binding, polymer matrices are preferred carriers. Polymer matrices useful in the invention include but are not limited to those matrices made of polyethylene glycol, polydextrans, cyclodextrins, polylysines, and the like. Variously sized polymer molecules can be evaluated to optimize attachment of a peptide conjugate and biodistribution following administration to a subject.

In one embodiment of the present invention, a polyethylene glycol (PEG) matrix is used. The term “polyethylene glycol” refers to straight or branched polyethylene glycol polymers and monomers. A PEG monomer is of the formula: —(CH₂CH₂O)—. Drugs and/or peptide analogs can be bound to PEG directly or indirectly, i.e. through appropriate spacer groups such as sugars. A PEG/peptide analog/drug composition can also include additional lipophilic and/or hydrophilic moieties to facilitate drug stability and delivery to a target site in vivo.

Peptides and drugs can be coupled to drugs or drug carriers using methods known in the art, including but not limited to carbodiimide conjugation, esterification, sodium periodate oxidation followed by reductive alkylation, and glutaraldehyde crosslinking. Representative methods for preparing PEG-containing compositions can be found in U.S. Pat. Nos. 6,461,603; 6,309,633; and 5,648,095, among other places.

II.F. Synthesis

Peptides of the present invention, including peptoids, can be synthesized by any of the techniques that are known to those skilled in the art of peptide synthesis. Synthetic chemistry techniques, such as a solid-phase Merrifield-type synthesis, are preferred for reasons of purity, antigenic specificity, freedom from undesired side products, ease of production and the like. A summary of representative techniques can be found in Stewart & Young (1984) Solid Phase Peptide Synthesis, Pierce Chemical Co., Rockville, Ill.; Merrifield (1969) Adv Enzymol Relat Areas Mol Biol 32:221-296; Fields & Noble (1990) Int J Pept Protein Res 35:161-214; Bodanszky (1993) Principles of Peptide Synthesis, Springer-Verlag, New York. Solid phase synthesis techniques can be found in Andersson et al. (2000) Biopolymers 55:227-50, references cited therein, and in U.S. Pat. Nos. 6,015,561, 6,015,881, 6,031,071, and 4,244,946. Peptides that include naturally occurring amino acids can also be produced using recombinant DNA technology. In addition, peptides comprising a specified amino acid sequence can be purchased from commercial sources (e.g., Biopeptide Co., LLC of San Diego, Calif. and PeptidoGenics of Livermore, Calif.).

A peptide mimetic is identified by assigning a hashed bitmap structural fingerprint to the peptide based on its chemical structure, and determining the similarity of that fingerprint to that of each compound in a broad chemical database. The fingerprints can be determined using fingerprinting software commercially distributed for that purpose by Daylight Chemical Information Systems, Inc. (Mission Viejo, Calif.) according to the vendor's instructions. Representative databases include but are not limited to SPREI'95 (InfoChem GmbH of München, Germany), Index Chemicus (ISI of Philadelphia, Pa.), World Drug Index (Derwent of London, United Kingdom), TSCA93 (United States Environmental Protection Agency), MedChem (Biobyte of Claremont, Calif.), Maybridge Organic Chemical Catalog (Maybridge of Cornwall, England), Available Chemicals Directory (MDL Information Systems of San Leandro, Calif.), NCI96 (United States National Cancer Institute), Asinex Catalog of Organic Compounds (Asinex Ltd. of Moscow, Russia), and NP (InterBioScreen Ltd. of Moscow, Russia). A peptide mimetic of a reference peptide is selected as comprising a fingerprint with a similarity (Tanamoto coefficient) of at least 0.85 relative to the fingerprint of A peptide mimetic can also be designed by: (a) identifying the pharmacophoric groups responsible for the targeting activity of a peptide; (b) determining the spatial arrangements of the pharmacophoric groups in the active conformation of the peptide; and (c) selecting a pharmaceutically acceptable template upon which to mount the pharmacophoric groups in a manner that allows them to retain their spatial arrangement in the active conformation of the peptide. For identification of pharmacophoric groups responsible for targeting activity, mutant variants of the peptide can be prepared and assayed for targeting activity. Alternatively or in addition, the three-dimensional structure of a complex of the peptide and its target molecule can be examined for evidence of interactions, for example the fit of a peptide side chain into a cleft of the target molecule, potential sites for hydrogen bonding, etc. The spatial arrangements of the pharmacophoric groups can be determined by NMR spectroscopy or X-ray diffraction studies. An initial three-dimensional model can be refined by energy minimization and molecular dynamics simulation. A template for modeling can be selected by reference to a template database and will typically allow the mounting of 2-8 pharmacophores. A peptide mimetic is identified wherein addition of the pharmacophoric groups to the template maintains their spatial arrangement as in the peptide. Techniques for the design and preparation of peptide mimetics can be found in U.S. Pat. Nos. 5,811,392; 5,811,512; 5,578,629; 5,817,879; 5,817,757; and 5,811,515.

Any peptide or peptide mimetic of the present invention can be used in the form of a pharmaceutically acceptable salt. Suitable acids which are capable of the peptides with the peptides of the present invention include inorganic acids such as trifluoroacetic acid (TFA), hydrochloric acid (HCl), hydrobromic acid, perchloric acid, nitric acid, thiocyanic acid, sulfuric acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid or the like. HCl and TFA salts are particularly preferred.

Suitable bases capable of forming salts with the peptides of the present invention include inorganic bases such as sodium hydroxide, ammonium hydroxide, potassium hydroxide and the like; and organic bases such as mono-di- and tri-alkyl and aryl amines (e.g. triethylamine, diisopropyl amine, methyl amine, dimethyl amine and the like), and optionally substituted ethanolamines (e.g. ethanolamine, diethanolamine and the like).

III. Uses of Peptide Analogs

Somatostatin analogs of the invention have utility in the detection of somatostatin receptors in vitro and in vivo, and in the diagnosis and treatment of SSTR-associated diseases and disorders. The term “somatostatin-associated,” as used herein to describe a disease or disorder treatable by the disclosed peptide analogs, refers to a condition characterized by abnormal SSTR expression and/or function. Abnormal SSTR expression refers to somatostatin receptor expression on the surface of a specific normal cell type, which expression is at a level significantly greater than a surface expression level normally associated with that specific normal cell type. For example, tumors characterized as neuroblastomas aberrantly express somatostatin receptors in that the cells of a neuroblastoma have a higher level of somatostatin receptor surface expression than the nerve tissue from which the neuroblastoma was derived. Abnormal SSTR function refers to conditions of abnormally elevated or abnormally suppressed signaling via SSTR. Such conditions are characterized, for example, by abnormal production of a somatostatin regulatable factor(s), which production is significantly greater than production of that same factor in the absence of the condition. Acromegaly, which is associated with over production of the somatostatin-regulatable factor, growth hormone and insulin-like growth factor-1, is an example of such a condition.

The utility of the disclosed peptide analogs relies on their ability to specifically bind cognate receptors. When administered to a subject, peptide analogs of the invention behave as targeting peptides. Thus, drugs bound to the peptide analogs can be delivered to specific cells in vivo.

The term “targeting” refers to the preferential movement and/or accumulation of a peptide or peptide analog in a target tissue as compared with a control tissue.

The term “target tissue” as used herein refers to an intended site for accumulation of a peptide analog following administration to a subject. For example, the methods of the present invention employ a target tissue comprising SSTR⁺ cells.

The term “control tissue” as used herein refers to a site suspected to substantially lack binding and/or accumulation of an administered peptide. For example, in accordance with the methods of the present invention, a control tissue that lacks SSTR⁺ cells, i.e., a tissue that is substantially SSTR⁻ cells, including SSTR⁻ cancer and non-cancer cells.

The term “selective targeting” is used herein to refer to a preferential localization of a peptide analog such that an amount of peptide analog in a target tissue is about 2-fold greater than an amount of peptide analog in a control tissue, more such as an amount that is about 5-fold or greater, or such as an amount that is about 10-fold or greater. The term “selective targeting” also refers to binding or accumulation of a peptide analog in a target tissue concomitant with an absence of targeting to a control tissue.

The term “cancer” refers to both primary and metastasized tumors and carcinomas of any tissue in a subject, including solid tumors arising from hematopoietic malignancies such as leukemias and lymphomas. In particular, somatostatin analogs of the present invention are useful for the treatment of neuroendocrine malignancies, as well as many other solid tumors, such as breast, lung, renal, pancreatic, gastric, colon, and brain. See e.g., Weckbecker et al. (1993) Pharmacol Ther 60:245-64; Srkalovic et al. (1990) J Clin Endocrinol Metab 70:661-9; Buscail et al. (1995) Proc Natl Acad Sci U S A 92:1580-4; Reubi et al. (1995) J Clin Endocrinol Metab 80:2806-14; Reubi et al. (1996) Metabolism 45:39-41; Buscail et al. (1994) Proc Natl Acad Sci USA 91:2315-9; Patel (1997) J Endocrinol Invest 20:348-67; Patel et al. (1995) Life Sci 57:1249-65; Bruns et al. (1994) Ann N Y Acad Sci 733:138-46; Reisine & Bell (1995) Endocr Rev 16:427-42; Krenning et al. (1993) Eur J Nucl Med 20:716-31; Plonowski et al. (2002) Int J Oncol 20:397-402; Szepeshazi et al. (2001) Clin Cancer Res 7:2854-61; Kiaris et al. (2001) Eur J Cancer 37:620-8; Plonowski et al. (2000) Cancer Res 60:2996-3001; Kahan et al. (1999) Int J Cancer 82:592-8; Plonowski et al. (1999) Cancer Res 59:1947-53.

The present invention also provides that the disclosed therapeutic and diagnostic methods can be used in combination. In addition, the disclosed methods can be used in combination with therapeutic and diagnostic methods known in the art. For example, peptide analogs of the invention can be administered for the dual purpose of detection and therapy.

III.A. Therapeutic Compositions and Methods

In another embodiment of the invention, a peptide analog comprising a therapeutic agent can be used to treat diseases or disorders characterized by cells that show abnormal of a receptor to which the targeting peptide specifically binds. Thus, also provided are methods for the treatment of SSTR-associated diseases and disorders. In a representative embodiment of the invention, the method comprises administering to a subject in need of such treatment a composition comprising a somatostatin analog of the formula (A-B), wherein A is cysteine, or a peptide chain comprising one or more cysteine residues, wherein a therapeutic agent is bound to A via thiol linkage to the one or more cysteine residues, and wherein B is a somatostatin peptide, whereby an SSTR-associated disease or disorder is treated.

The somatic analogs disclosed herein can be used to inhibit secretion of growth hormone, somatomedins (e.g., IGF-1), insulin, glucagon, and other autoparacrine growth factors or pancreatic growth factors. Thus, the compounds of the invention can be used to treat disorders resulting from growth hormone overproduction, such as, for the treatment of acromegaly and/or type II diabetes. See e.g., Jenkins et al. (2001) Chemotherapy 47 Suppl 2:162-96.

For the treatment of cancer, the somatostatin analogs of the invention are bound to an anti-cancer drug, including but not limited to radioisotopes, cytotoxins (e.g., a tubulin inhibitor), therapeutic genes, immunostimulatory agents, anti-angiogenic agents, and chemotherapeutic agents. Representative members of these drug types, which are not mutually exclusive, are summarized herein below. Administration of a somatostatin analog of the invention may elicit an anti-tumor response, such as inhibition of tumor growth. See Examples 4-5.

For radiotherapy applications, a peptide analog of the invention can comprise a high energy radioisotope bound to the analog at a free cysteine. The isotope can be directly bound at a cysteine residue present in the peptide, or the binding can include the use of a chelator which is bound to the peptide analog via a thiol-specific linkage. Radioisotopes suitable for radiotherapy include but are not limited to α-emitters, β-emitters, and auger electrons. Representative radioisotopes include ¹⁸fluorine, ⁶⁴copper, ⁶⁵copper, ⁶⁷gallium, ⁶⁸gallium, ⁷⁷bromine, ^{80m}bromine, ⁹⁵ruthenium, ⁹⁷ruthenium, ¹⁰³ruthenium, ¹⁰⁵ruthenium, ^{99m}technetium, ¹⁰⁷mercury, ²⁰³mercury, ¹²³iodine, ¹²⁴iodine, ¹²⁵iodine, ¹²⁶iodine, ¹³¹iodine, ¹³³iodine ¹¹¹Indium, ¹¹³mindium, ^{99m}rheniu , ¹⁰⁵rhenium, ¹⁰¹rhenium, ¹⁸⁶rhenium, ¹⁸⁸rhenium, ^{121m}tellurium, ⁹⁹technetium, ^{122m}tellurium, ^{125m}tellurium, ¹⁶⁵thulium, ¹⁶⁷thulium, ¹⁶⁸thulium, ⁹⁰yttrium, and nitride or oxide forms derived there from. Other suitable radioisotopes include alpha emitters, such as ²¹³bismuth, ²¹³lead, and ²²⁵actinium.

Methods for radioisotope-labeling of a molecule so as to be used in accordance with the disclosed methods are known in the art. For example, a targeting molecule can be derivatized so that a radioisotope can be bound directly to it (Yoo et al., 1997). Alternatively, a linker can be added that to enable conjugation. Representative linkers include diethylenetriamine pentaacetate (DTPA)-isothiocyanate, succinimidyl 6-hydrazinium nicotinate hydrochloride (SHNH), and hexamethylpropylene amine oxime (HMPAO). See Chattopadhyay et al. (2001) Nucl Med Biol 28: 741-4; Dewanjee et al. (1994) J Nucl Med 35: 1054-63; Sagiuchi et al. (2001) Ann Nucl Med 15: 267-70; U.S. Pat. No. 6,024,938. See also Example 1.

Angiogenesis and suppressed immune response play a central role in the pathogenesis of malignant disease and tumor growth, invasion, and metastasis. Thus, drugs useful in the methods of the present invention also include those able to induce an immune response and/or an anti-angiogenic response in vivo.

The term “immune response” is meant to refer to any response to an antigen or antigenic determinant by the immune system of a vertebrate subject. Exemplary immune responses include humoral immune responses (e.g. production of antigen-specific antibodies) and cell-mediated immune responses (e.g. lymphocyte proliferation),

Representative therapeutic proteins with immunostimulatory effects include but are not limited to cytokines (e.g., IL2, IL4, IL7, IL12, interferons, granulocyte-macrophage colony-stimulating factor (GM-CSF), tumor necrosis factor alpha (TNF-α)), immunomodulatory cell surface proteins (e.g., human leukocyte antigen (HLA proteins), co-stimulatory molecules, and tumor-associated antigens. See Kirk & Mule (2000) Hum Gene Ther 11:797-806; Mackensen et al. (1997) Cytokine Growth Factor Rev 8:119-128; Walther & Stein (1999) Mol Biotechnol 13:21-28; and references cited therein.

The term “angiogenesis” refers to the process by which new blood vessels are formed. The term “anti-angiogenic response” and “anti-angiogenic activity” as used herein, each refer to a biological process wherein the formation of new blood vessels is inhibited.

Representative proteins with anti-angiogenic activities that can be used in accordance with the present invention include: thrombospondin I (Dameron et al. (1994) Science 265: 1582-4; Kosfeld et al. (1993) J Biol Chem 268: 8808-14; Tolsma et al. (1993) J Cell Biol 122: 497-511), metallospondin proteins (Carpizo et al. (2000) Cancer Metastasis Rev 19: 159-65), class I interferons (Albini et al. (2000) Am J Pathol 156: 1381-93), IL12 (Voest et al. (1995) J Natl Cancer Inst 87: 581-6), protamine (Ingber et al. (1990) Nature 348: 555-7), angiostatin (O'Reilly et al. (1994) Cell 79: 315-28), laminin (Sakamoto et al. (1991) Cancer Res 51: 903-6), endostatin (O'Reilly et al. (1997) Cell 88: 277-85), and a prolactin fragment (Clapp et al. (1993) Endocrinology 133: 1292-9). In addition, several anti-angiogenic peptides have been isolated from these proteins (Eijan et al. (1991) Mol Biother 3: 38-40; Maione et al. (1990) Trends Pharmacol Sci 11: 457-61; Woltering et al. (1991) J Surg Res 50: 245-51).

Additional anti-tumor agents that can be conjugated to the somatostatin analogs disclosed herein and used in accordance with the therapeutic methods of the present invention include but are not limited to alkylating agents such as melphalan and chlorambucil, vinca alkaloids such as vindesine and vinblastine, antimetabolites such as 5-fluorouracil, 5-fluorouridine and derivatives thereof. See e.g., Aboud-Pirak et al. (1989) Biochem Pharmacol 38: 641-8; Rowland et al. (1993) Cancer Immunol Immunother 37: 195-202; Smyth et al. (1987) Immunol Cell Biol 65 (Pt 4): 315-21; Starling et al. (1992) Bioconjug Chem 3: 315-22; Krauer et al. (1992) Cancer Res 52: 132-7; Henn et al. (1993) J Med Chem 36: 1570-9.

The somatostatin analogs disclosed herein can be combined with other therapies, including but not limited to chemotherapy, surgical excision, radiation, radiosensitization, chemoprotection, anti-angiogenic treatment, immunostimulatory treatments, gene therapy, and hormonal therapy. The combination therapy can elicit additive or potentiated therapeutic effects and/or reduce hepatotoxicity of some anti-cancer agents. See e.g., Davies et al. (1996) Anticancer Drugs 7 Suppl 1:23-31; Lee et al. (1993) Anticancer Res 13:1453-6; Stewart et al. (1994) Br J Surg 81:1332.

III.B. Diagnostic and Detection Methods

The present invention further provides methods whereby a peptide analog comprising a detectable label can be used to detect the presence of cells having a receptor that specifically binds the targeting peptide. The methods are applicable to in vitro and in vivo detection.

In one embodiment of the invention, a method for detecting SSTR-expressing cells can comprise: (a) preparing a biological sample comprising cells; (c) contacting a somatostatin analog of the invention with the biological sample in vitro, wherein the somatostatin analog comprises a detectable label; and (c) detecting the detectable label, whereby SSTR-expressing cells are detected. For example, peptide conjugates of the invention can be used to detect and quantify SSTR-positive cells or tissues.

In another embodiment of the invention, the disclosed detection methods are performed in vivo, for example as useful for diagnosis or to provide intraoperative assistance. Thus, the detection method of the present invention can also comprise: (a) administering to the subject a composition comprising a somatostatin analog of the formula (A-B), wherein A is cysteine, or a peptide chain comprising one or more cysteine residues, wherein A is bound to the one or more cysteines via a thiol linkage, and wherein B is a somtaostatin peptide; and (b) detecting the detectable label, whereby SSTR-positive cells are detected.

Following administration of a labeled peptide analog to a subject, and after a time sufficient for binding, the biodistribution of the composition can be visualized. The term “time sufficient for binding” refers to a temporal duration that permits binding of the peptide analog to cognate receptors in vivo.

The term “in vivo”, as used herein to describe imaging or detection methods, refer to generally non-invasive methods such as scintigraphic methods, magnetic resonance imaging, ultrasound, or fluorescence, each described briefly herein below. The term “non-invasive methods” does not exclude methods employing administration of a contrast agent to facilitate in vivo imaging. For in vitro detection, useful detectable labels include a fluorophore, an epitope, or a radioactive label, also described briefly herein below.

Scintigraphic Imaging. For detection of SSTR-expressing cells by scintigraphy, a somatostatin analog of the invention is prepared by thiol-specific attachment of a radioisotope to the analog. Diagnostic radioisotopes include but are not limited to γ-emitters and positron emitters. Representative methods for preparing a radioisotope-labeled agent are described herein above. Stabilizers to prevent or minimize radiolytic damage, such as ascorbic acid, gentisic acid, or other appropriate antioxidants, can be added to the composition comprising the labeled peptide analog.

Scintigraphic imaging methods include SPECT (Single Photon Emission Computed Tomography), PET (Positron Emission Tomography), gamma camera imaging, and rectilinear scanning. A gamma camera and a rectilinear scanner each represent instruments that detect radioactivity in a single plane. Most SPECT systems are based on the use of one or more gamma cameras that are rotated about the subject of analysis, and thus integrate radioactivity in more than one dimension. PET systems comprise an array of detectors in a ring that also detect radioactivity in multiple dimensions.

Imaging instruments suitable for practicing the method of the present invention, and instruction for using the same, are readily available from commercial sources. Both PET and SPECT systems are offered by ADAC of Milpitas, Calif. and Siemens of Hoffman Estates, Ill. Related devices for scintigraphic imaging can also be used, such as a radio-imaging device that includes a plurality of sensors with collimating structures having a common source focus. Magnetic Resonance Imaging (MRI). Magnetic resonance image-based techniques create images based on the relative relaxation rates of water protons in unique chemical environments. As used herein, the term “magnetic resonance imaging” refers to magnetic source techniques including convention magnetic resonance imaging, magnetization transfer imaging (MTI), proton magnetic resonance spectroscopy (MRS), diffusion-weighted imaging (DWI) and functional MR imaging (fMRI). See Rovaris et al. (2001) J Neurol Sci 186 Suppl 1:S3-9; Pomper & Port (2000) Magn Reson Imaging Clin N Am 8:691-713; and references cited therein.

Contrast agents for magnetic source imaging include but are not limited to paramagnetic or superparamagnetic ions, iron oxide particles (Shen et al., 1993; Weissleder et al., 1992), and water soluble contrast agents. Paramagnetic and superparamagnetic ions can be selected from the group of metals including iron, copper, manganese, chromium, erbium, europium, dysprosium, holmium and gadolinium. Preferred metals are iron, manganese and gadolinium; most preferred is gadolinium.

Those skilled in the art of diagnostic labeling recognize that metal ions can be bound by chelating moieties, which in turn can be conjugated to a therapeutic agent in accordance with the methods of the present invention. For example, gadolinium ions are chelated by diethylenetriaminepentaacetic acid (DTPA). Lanthanide ions are chelated by tetraazacyclododocane compounds. See U.S. Pat. Nos. 5,738,837 and 5,707,605. Alternatively, a contrast agent can be carried in a liposome (Schwendener, 1992).

Images derived used a magnetic source can be acquired using, for example, a superconducting quantum interference device magnetometer (SQUID, available with instruction from Quantum Design of San Diego, Calif.). See U.S. Pat. No. 5,738,837.

Ultrasound. Ultrasound imaging can be used to obtain quantitative and structural information of a target tissue, including a tumor. Administration of a contrast agent, such as gas microbubbles, can enhance visualization of the target tissue during an ultrasound examination. The contrast agent can be selectively targeted to the target tissue of interest, for example by using a peptide for x-ray guided drug delivery as disclosed herein. Representative agents for providing microbubbles in vivo include but are not limited to gas-filled lipophilic or lipid-based bubbles (e.g., U.S. Pat. Nos. 6,245,318, 6,231,834, 6,221,018, and 5,088,499). In addition, gas or liquid can be entrapped in porous inorganic particles that facilitate microbubble release upon delivery to a subject (U.S. Pat. Nos. 6,254,852 and 5,147,631).

Gases, liquids, and combinations thereof suitable for use with the invention include air; nitrogen; oxygen; is carbon dioxide; hydrogen; nitrous oxide; an inert gas such as helium, argon, xenon or krypton; a sulphur fluoride such as sulphur hexafluoride, disulphur decafluoride or trifluoromethylsulphur pentafluoride; selenium hexafluoride; an optionally halogenated silane such as tetramethylsilane; a low molecular weight hydrocarbon (e.g. containing up to 7 carbon atoms), for example an alkane such as methane, ethane, a propane, a butane or a pentane, a cycloalkane such as cyclobutane or cyclopentane, an alkene such as propene or a butene, or an alkyne such as acetylene; an ether; a ketone; an ester; a halogenated low molecular weight hydrocarbon (e.g. containing up to 7 carbon atoms); or a mixture of any of the foregoing. Halogenated hydrocarbon gases can show extended longevity, and thus are preferred for some applications. Representative gases of this group include decafluorobutane, octafluorocyclobutane, decafluoroisobutane, octafluoropropane, octafluorocyclopropane, dodecafluoropentane, decafluorocyclopentane, decafluoroisopentane, perfluoropexane, perfluorocyclohexane, perfluoroisohexane, sulfur hexafluoride, and perfluorooctaines, perfluorononanes, perfluorodecanes, optionally brominated.

Attachment of peptide analogs to lipophilic bubbles can be accomplished via chemical crosslinking agents in accordance with standard protein-polymer or protein-lipid attachment methods (e.g., via carbodiimide (EDC) or thiopropionate (SPDP)). To improve targeting efficiency, large gas-filled bubbles can be coupled to a peptide analog using a flexible spacer arm, such as a branched or linear synthetic polymer (U.S. Pat. No. 6,245,318). A peptide analog can be attached to the porous inorganic particles by coating, adsorbing, layering, or reacting the outside surface of the particle with the peptide analog (U.S. Pat. No. 6,254,852).

A description of ultrasound equipment and technical methods for acquiring an ultrasound dataset can be found in Coatney (2001) ILAR J 42:233-247, Lees (2001) Semin Ultrasound CT MR 22:85-105, and references cited therein.

Fluorescent Imaging. Non-invasive imaging methods can also comprise detection of a fluorescent label. A drug comprising a lipophilic component (therapeutic agent, diagnostic agent, vector, or drug carrier) can be labeled with any one of a variety of lipophilic dyes that are suitable for in vivo imaging. See e.g. Fraser (1996) Methods Cell Biol 51:147-160; Ragnarson et al. (1992) Histochemistry 97:329-333; and Heredia et al. (1991) J Neurosci Methods 36:17-25. Representative labels include but are not limited to carbocyanine and aminostyryl dyes, such as long chain dialkyl carbocyanines (e.g., DiI, DiO, and DiD available from Molecular Probes Inc. of Eugene, Oreg.) and dialkylaminostyryl dyes. Lipophilic fluorescent labels can be incorporated using methods known to one of skill in the art. For example VYBRANT™ cell labeling solutions are effective for labeling of cultured cells of other lipophilic components (Molecular Probes Inc. of Eugene, Oreg.).

A fluorescent label can also comprise sulfonated cyanine dyes, including CyS.5 and Cy5 (available from Amersham of Arlington Heights, Ill.), IRD41 and IRD700 (available from Li-Cor, Inc. of Lincoln, Nebr.), NIR-1 (available from Dejindo of Kumamoto, Japan), and LaJolla Blue (available from Diatron of Miami, Fla.). See also Licha et al. (2000) Photochem Photobiol 72:392-398; Weissleder et al. (1999) Nat Biotechnol 17:375-378; and Vinogradov et al. (1996) Biophys J 70:1609-1617.

In addition, a fluorescent label can comprise an organic chelate derived from lanthanide ions, for example fluorescent chelates of terbium and europium (U.S. Pat. No. 5,928,627). Such labels can be conjugated or covalently linked to a drug as disclosed therein.

For in vivo detection of a fluorescent label, an image is created using emission and absorbance spectra that are appropriate for the particular label used. The image can be visualized, for example, by diffuse optical spectroscopy. Additional methods and imaging systems are described in U.S. Pat. Nos. 5,865,754; 6,083,486; and 6,246,901, among other places.

Fluorescence. Any detectable fluorescent dye can be used, including but not limited to FITC (fluorescein isothiocyanate), FLUOR X™, ALEXA FLUOR®, OREGON GREEN®, TMR (tetramethylrhodamine), ROX (X-rhodamine), TEXAS RED®, BODIPY® 630/650, and Cy5 (available from Amersham Pharmacia Biotech of Piscataway, N.J. or from Molecular Probes Inc. of Eugene, Oreg.).

A fluorescent label can be detected directly using emission and absorbance spectra that are appropriate for the particular label used. Common research equipment has been developed for in vitro detection of fluorescence, including instruments available from GSI Lumonics (Watertown, Mass., United States of America) and Genetic MicroSystems Inc. (Woburn, Mass., United States of America). Most of the commercial systems use some form of scanning technology with photomultiplier tube detection.

Detection of an Epitope. If an epitope label has been used, a protein or compound that binds the epitope can be used to detect the epitope. A representative epitope label is biotin, which can be detected by binding of an avidin-conjugated fluorophore, for example avidin-FITC, as described in Example 7. Alternatively, the label can be detected by binding of an avidin-horseradish peroxidase (HRP) streptavidin conjugate, followed by colorimetric detection of an HRP enzymatic product. The production of a colorimetric or luminescent product/conjugate is measurable using a spectrophotometer or luminometer, respectively.

Autoradiographic Detection. In the case of a radioactive label detection can be accomplished by conventional autoradiography or by using a phosphorimager as is known to one of skill in the art. A preferred autoradiographic method employs photostimulable luminescence imaging plates (Fuji Medical Systems of Stamford, Conn.). Briefly, photostimulable luminescence is the quantity of light emitted from irradiated phosphorous plates following stimulation with a laser during scanning. The luminescent response of the plates is linearly proportional to the activity.

III.C In Vivo Methods

The compositions of the invention can be formulated according to known methods to prepare pharmaceutical compositions. Suitable formulations for administration to a subject include aqueous and non-aqueous sterile injection solutions which can contain anti-oxidants, buffers, bacteriostats, antibacterial and antifungal agents (e.g., parabens, chlorobutanol, phenol, ascorbic acid, an thimerosal), solutes that render the formulation isotonic with the bodily fluids of the intended recipient (e.g., sugars, salts, and polyalcohols), suspending agents and thickening agents. Suitable solvents include water, ethanol, polyol (e.g., glycerol, propylene glycol, and liquid polyethylene glycol), and mixtures thereof. The formulations can be presented in unit-dose or multi-dose containers, for example sealed ampoules and vials, and can be stored in a frozen or freeze-dried (lyophilized) condition requiring only the addition of sterile liquid carrier immediately prior to use for administration to a subject or for subsequent radiolabeling with an isotope appropriate for the intended application.

The formulations according to the invention are buffered to a pH of from about 5 to about 7, or about 6. Suitable buffers are those which are physiologically acceptable upon administration by inhalation. Such buffers include citric acid buffers and phosphate buffers, of which phosphate buffers are preferred. Particularly preferred buffers for use in the formulations of the invention are monosodium phosphate dihydrate and dibasic sodium phosphate anhydrous.

Suitable methods for administration of peptide analogs include but are not limited to intravascular, subcutaneous, or intratumoral administration. For delivery of compositions to pulmonary pathways, compositions can be administered as an aerosol or coarse spray.

To minimize renal uptake of a peptide analog, an amino acid infusion can be administered prior to administration of the analog. See e.g., Hammond et al. (1993) Br J Cancer 67:1437-9 and U.S. Pat. No. 6,277,356.

The present invention provides that an effective amount of a peptide analog is administered to a subject. The term “effective amount” is used herein to describe an amount of a peptide analog sufficient to elicit a desired biological response. For example, when administered to a cancer-bearing subject, an effective amount comprises an amount sufficient to elicit an anti-cancer activity, including cancer cell cytolysis, inhibition of cancer growth, inhibition of cancer metastasis, and/or cancer resistance. Typical dosages of a radioisotope or peptide analog are from about 0.1 pg/kg to 500 μg/kg, or about 1 ng/kg to 500 μg/kg, or about 200 ng/kg, depending on the specific activity of the radioisotope attached to the peptide. Alternatively, the analog can be administered at a dosage range having an amount of radioactivity of from about 10 μCi/kg to 5 mCi/kg body weight. Generally, the total amount of radioisotope delivered in a single dose is from about 1 mCi to about 300 mCi, normally about 5 mCi to 100 mCi, depending on the radioisotope and the specific activity of the targeting peptide.

For diagnostic applications, a detectable amount of a composition of the invention is administered to a subject. A “detectable amount,” as used herein to refer to a diagnostic composition, refers to a dose of a peptide analog such that the presence of the analog can be determined in vivo following administration to the subject. For scintigraphic imaging using radioisotopes, a detectable dose can include doses within a range defined by a bell-shaped curve. See e.g., Breeman et al. (1999) Int J Cancer 81:658-65. In general, typical doses of a radioisotope can include an activity of about 10 μCi to 50 mCi, or about 100 μCi to 25 mCi, or about 500 μCi to 20 mCi, or about 1 mCi to 10 mCi, or about 10 mCi.

Actual dosage levels of active ingredients in a composition of the invention can be varied so as to administer an amount of the composition that is effective to achieve the desired diagnostic or therapeutic outcome for a particular subject. Administration regimens can also be varied. A single injection or multiple injections can be used. The selected dosage level and regimen will depend upon a variety of factors including the activity of the therapeutic composition, formulation, the route of administration, combination with other drugs or treatments, the disease or disorder to be detected and/or treated, and the physical condition and prior medical history of the subject being treated. Determination and adjustment of an effective amount or dose, as well as evaluation of when and how to make such adjustments, are known to those of ordinary skill in the art of medicine. For example, a minimal dose is administered, and dose is escalated in the absence of dose-limiting toxicity. Determination and adjustment of a therapeutically effective dose, as well as evaluation of when and how to make such adjustments, are known to those of ordinary skill in the art of medicine.

For additional guidance regarding formulation, dose and administration regimen, see Berkow et al. (2000) The Merck Manual of Medical Information, Merck & Co., Inc., Whitehouse Station, N.J.; Ebadi (1998) CRC Desk Reference of Clinical Pharmacology, CRC Press, Boca Raton, Fla.; Gennaro (2000) Remington: The Science and Practice of Pharmacy, Lippincott, Williams & Wilkins, Philadelphia, Pa.; Katzung (2001) Basic & Clinical Pharmacology, Lange Medical Books/McGraw-Hill Medical Pub. Div., New York; Hardman et al. (2001) Goodman & Gilman's the Pharmacological Basis of Therapeutics, The McGraw-Hill Companies, Columbus, Ohio; Speight & Holford (1997) Avery's Drug Treatment: A Guide to the Properties, Choices Therapeutic Use and Economic Value of Drugs in Disease Management, Lippincott, Williams, & Wilkins, Philadelphia, Pa.

### EXAMPLES

The following Examples have been included to illustrate modes of the invention. Certain aspects of the following Examples are described in terms of techniques and procedures found or contemplated by the present co-inventors to work well in the practice of the invention. These Examples illustrate standard laboratory practices of the co-inventors. In light of the present disclosure and the general level of skill in the art, those of skill will appreciate that the following Examples are intended to be exemplary only and that numerous changes, modifications, and alterations can be employed without departing from the scope of the invention.

### Example 1

### Preparation of Peptide Conjugates

The CP1-AEBL conjugate was prepared using a maleimido derivative of Auristatin E (AEBL) reacted via the thiol of the free cysteine of CP1. The chemistry provides an acid-labile hydrazone linkage that selectively releases AEB, a structural variant of AE having similar potency.

The CPI-FKMMAE conjugate was prepared using a derivative of AE (FKMMAE) reacted via the thiol of the free CP1 cysteine. The FKMMAE drug structure contains a peptide linkage that is cleaved selectively by the intracellular enzyme cathepsin B. The drug released within the cell is a monomethyl derivative of AE and has potency similar to AE.

The CP1-chelator conjugate was prepared using a maleimido derivative of MX-DTPA, a high affinity chelator of Indium-111. MEM-MX-DTPA was incubated with CP1 at a 25% molar excess for 1.5 hours at room temperature. pH was neutral upon dilution of reactants with 100 mM phosphate containing 150M NaCl (70%) and DMF (30%). The reaction product was separated from reactants using HPLC by applying the reaction mixture to a C18 reverse phase column in a 25-35% gradient run over 60 minutes. Product elution was monitored at 215 nm and at 280 nm, and fractions were collected at 24-32 minutes, which period spanned potential product peaks. Fractions were identified using mass spectrometry. Fractions containing the CP1-MX-DTPA product were pooled, lyophilized using a speed vacuum, and stored at −70° C.

### Example 2

### Binding Affinity of Peptide Conjugate to Receptor

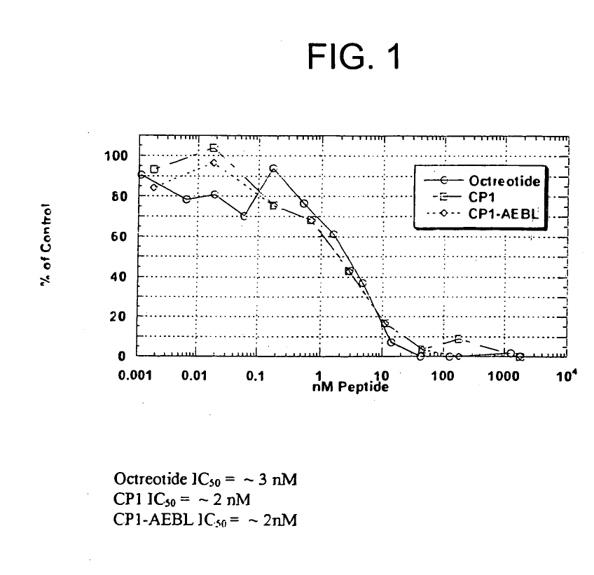

Affinity measurements of CP1-AEB binding were determined by performing a competition binding assay. The assay used partially purified membrane extracts from IMR-32 cells, a human neuroblastoma cell line expressing SSTR2. CP1-AEB, CP1 and Octreotide were titrated onto IMR-32 membranes in triplicate dilution tubes arranged in a 96-well plate format. Indium-111-Octreotide competitor was added to IMR-32 membranes, for 1 hour at room temperature in a diluent at neutral pH consisting of 10 mM Hepes, 1 mM MgCl₂, 0.3% BSA, and EDTA-free protease inhibitors. IMR-32 membranes were collected under vacuum onto glass fiber filter paper in a 96-well plate format, and membranes were washed four times with 10 mM Tris, 150 mM NaCl, pH 7.5. Captured membranes from each replicate were “punched out” into tubes for counting gamma radioactivity. To estimate an IC₅₀ of Indium-111-Octreotide binding to IMR-32 membranes, recovered radioactivity when using each competitor sample was expressed as a percent of the control sample (in the absence of competitor). See FIG. 1.

### Example 3

### Cellular Uptake of Peptide Conjugates

SSTR-positive cell lines (human neuroblastoma IMR-32, rat pancreatic carcinoma AR42J) or negative control cells (human colon adenocarcinoma LS174T) were incubated in 6-well plates overnight at 37° C. in a humidified incubator containing 5% C0₂. Approximately 10⁶ cpm of Indium-111-Octreotide or Indium-111-CP1-MX-DTPA was applied to triplicate wells, in a cocktail containing peptide plus 1000 molar excess somatostatin. Plates were again incubated overnight (20-24 hours) at 37° C. in a humidified incubator containing 5% CO₂. Cells were washed with PBS, trypsinized, and collected. Radioactivity present in the cell samples was counted to determine the amount of applied Indium-111-labeled peptide taken up by the cells. Percent uptake of applied cpm was calculated for each triplicate set of wells. Uptake of both peptides was specific, as indicated by the significant reduction in counts in the presence of excess somatostatin (Table 1).

TABLE 1
In Vitro Uptake of Indium-111-Labeled SST Peptides
by SSTR⁺ Human Cancer Cells

| | Percentage (%) | Percentage (%) Uptake in the |
|---|---|---|
| Peptide | Uptake | Presence of 1000X SST |
| ¹¹¹In-CP1-MX-DTPA | 1.9 | 0.2 |
| ^{Indium-111}-octreotide | 4.4 | 0.1 |

SSTR-positive rat pancreatic carcinoma cells (AR42J cells) also showed specific uptake of Indium-111-CP1-MX-DTPA, while SSTR-negative human colon carcinoma cells (LS174T cells) did not (Table 2).

TABLE 2
In Vitro Uptake of Indium-111-Labeled CP1-MX-DTPA
by SSTR⁺ Cancer Cells

| | | Percentage (%) Uptake in the |
|---|---|---|
| Cell Line | Percentage (%) Uptake | Presence of 1000X SST |
| IMR-32 (SSRT⁺) | 3.2 | 0.2 |
| AR42J (SSRT⁺) | 13.9 | 0.3 |
| LS174T (SSRT⁻) | 0.2 | 0.2 |

### Example 4

### Cytotoxicity Induced by Auristatin Peptide Conjugates

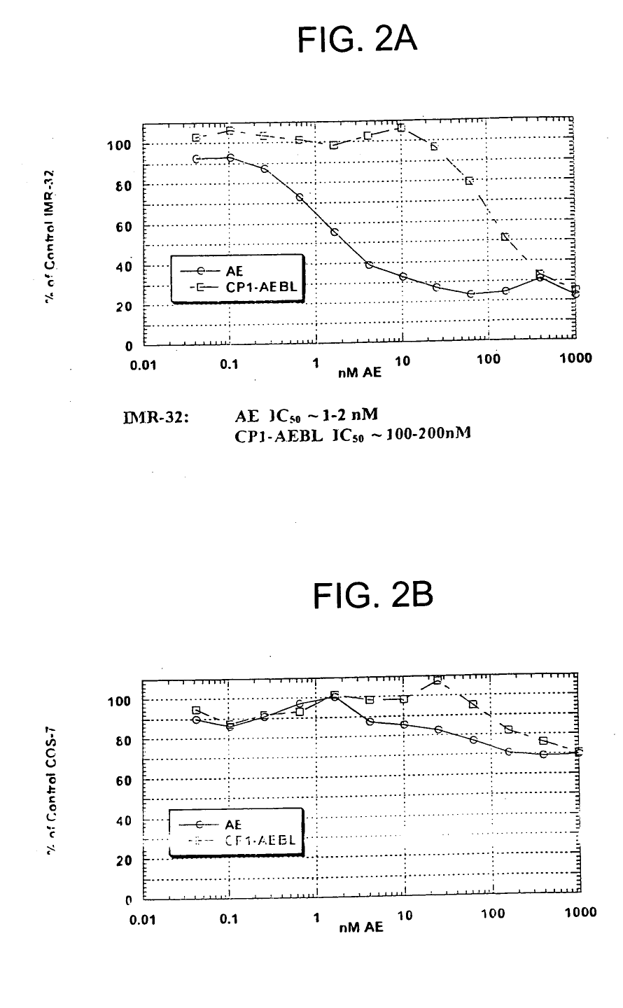

Approximately 50,000 SSTR-positive IMR-32 cells and SSTR-negative COS-7 cells were applied to each well of a 96-well plate. Cells were incubated overnight at 37° C. in a humidified incubator containing 5% CO₂. CP1-AEB was titrated into wells containing IMR-32 and COS-7 cells, in triplicate. Following incubation for 3-4 hours, the plated cells were washed and fresh media was applied. The plates were incubated an additional 48 hours before analysis of CP1-AEB toxicity. MTT (3-[4,5-dimethylthiazol-2-yl]-2,5-diphenyl-tetrazoliumbromide) was applied to the cells, and the cells were incubated in the presence of MTT for 3 to 4 hours. Levels of MTT uptake by live cells was measured calorimetrically for comparison between the two cell lines. See FIGS. 2A-2B.

### Example 5

### In Vivo Anti-Tumor Activity

The tumoricidal effects of CP1-FKMMAE were evaluated in a mouse xenograft model. Tumors were established in nude mice by subcutaneous injection of IMR-32 cells. A multiple dose regimen was evaluated based on prior studies using AE, which determined a MTD following four administrations of 0.4 mg/kg. Due to limited availability, AE was used at 75% of the MTD. CP1-FKMMAE was administered at 1× and 3× molar equivalents of AE according to the same dosing schedule. Tumor volume, animal weight, and serum growth hormone levels were assessed for each treatment group.

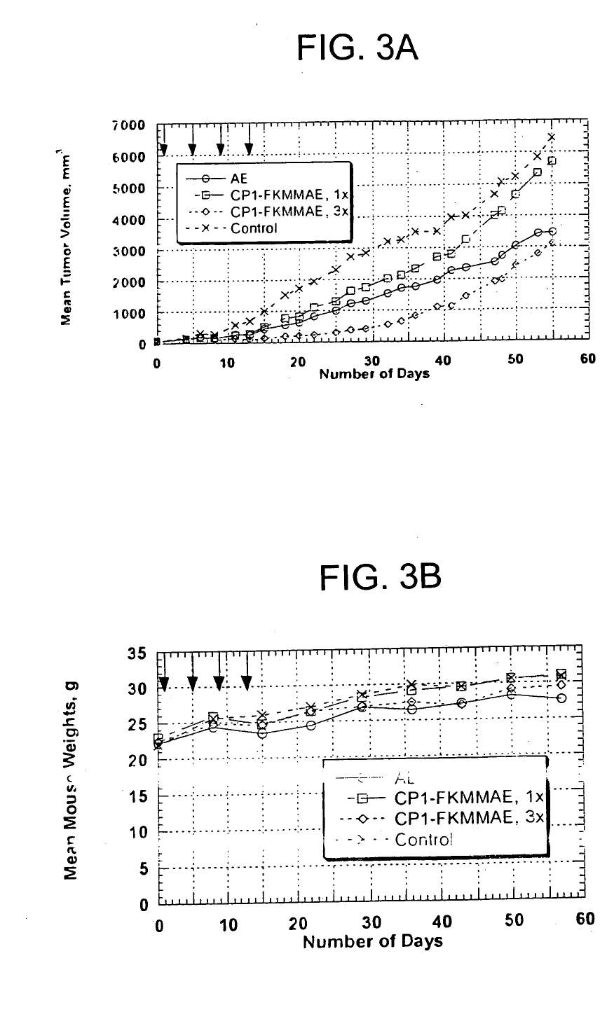

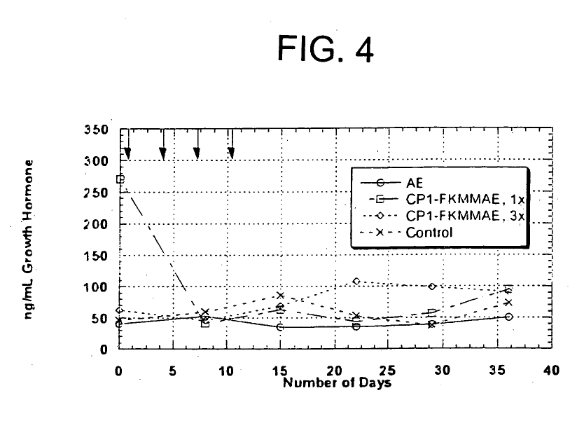

Inhibition of tumor growth was observed in all treatment groups, and animals receiving a 3× dose of CP1-FKMMAE showed the greatest level of tumor growth inhibition (FIGS. 3A-3B). All treatment groups showed an increase in animal weight and stable growth hormone levels (FIG. 3B and FIG. 4, respectively), suggesting that the MTD was not achieved.

While the present invention has been described in connection with what is presently considered to be practical and preferred embodiments, it is understood that the present invention is not to be limited or restricted to the disclosed embodiments but, on the contrary, is intended to cover various modifications and equivalent arrangements included within the scope of the appended claims. Thus, it is to be understood that variations in the described invention will be obvious to those skilled in the art without departing from the novel and non-obvious aspects of the present invention, and such variations are intended to come within the scope of the claims below.

1. A composition comprising a somatostatin analog of the formula:
(A−B)
wherein:
A is cysteine, or a peptide chain comprising one or more cysteine residues, which is suitable for binding to a drug or chelator via a thiol linkage; and
B is a naturally occurring or synthetic somatostatin peptide, or fragment thereof, that binds to a somatostatin receptor.

2. The composition of claim 1, wherein A comprises the peptide sequence of any one of SEQ ID NOs:1-3, or wherein A is a single cysteine residue.

3. The composition of claim 1, wherein B comprises SEQ ID NO:4.

4. The composition of claim 1, wherein the somatostatin analog comprises a peptide of any one of SEQ ID NOs:5-7.

5. The composition of claim 1, further comprising a drug, or chelator suitable for binding a drug, wherein the drug or chelator is bound to the one or more cysteines by a thiol linkage.

6. The composition of claim 5, wherein the drug is a therapeutic agent or a detectable label.

7. The composition of claim 6, wherein the therapeutic agent is a radioisotope, a cytotoxin, an immunostimulatory agent, an anti-angiogenic agent, a therapeutic gene, or a chemotherapeutic agent.

8. The composition of claim 5, wherein the chelator is a maleimido derivative of DTPA or a maleimido derivative of a DTPA analog.

9. The composition of claim 1, further comprising a somatostatin analog that specifically binds to mammalian SSTR-positive cells in vivo.

10. The composition of claim 1, wherein the SSTR-positive cells are human cancer cells.

11. A method for detecting SSTR-positive cells in a mammalian subject comprising:
(a) administering to the subject a composition comprising a somatostatin analog of the formula:
(A−B)
wherein:
A is cysteine, or a peptide chain comprising one or more cysteine residues, wherein a detectable label is bound to the one or more cysteine residues via a thiol linkage; and
B is a naturally occurring or synthetic somatostatin peptide, or fragment thereof, which binds to a somatostatin receptor; and
(b) detecting the detectable label, whereby SSTR-positive cells are detected.

12. The method of claim 11, wherein A comprises the peptide sequence of any one of SEQ ID NOs:1-3, or wherein A is a single cysteine residue.

13. The method of claim 11, wherein B comprises SEQ ID NO:4.

14. The method of claim 11, wherein the somatostatin analog comprises a peptide of any one of SEQ ID NOs:5-7.

15. A method for treating an SSTR-associated disorder in a mammalian subject, the method comprising administering to the subject a composition comprising a somatostatin analog of the formula:
(A−B)
wherein:
A is cysteine, or a peptide chain comprising one or more cysteine residues, wherein a therapeutic agent is bound to the one or more cysteine residues via a thiol linkage; and
B is a naturally occurring or synthetic somatostatin peptide, or fragment thereof, which binds to a somatostatin receptor; and
whereby a SSTR-associated disorder is treated.

16. The method of claim 15, wherein A comprises the peptide sequence of any one of SEQ ID NOs:1-3, or wherein A is a single cysteine residue.

17. The method of claim 15, wherein B comprises SEQ ID NO:4.

18. The method of claim 15, wherein the somatostatin analog comprises a peptide of any one of SEQ ID NOs:5-7.

19. The method of claim 15, wherein the therapeutic agent is selected from the group consisting of a radioisotope, a cytotoxin, an immunostimulatory agent, an anti-angiogenic agent, a therapeutic gene, and a chemotherapeutic agent.

20. The method of claim 15, wherein the SSTR-associated disorder is cancer.
